(19) United States
(12) Patent Application Publication
(10) Pub. No.: US 2010/0305213 A1
(43) Pub. Date: Dec. 2, 2010

(54) TREATMENT OF HEART DISEASE USING BETA-BLOCKERS

(75) Inventors: Gerald Beddies, Leverkusen (DE); Axel Schmidt, Wuppertal (DE)

Correspondence Address: BAYER HEALTHCARE LLC, P.O.BOX 390, SHAWNEE MISSION, KS, 66201, US

(73) Assignee: Bayer Animal Health GmbH, Leverkusen (DE)

(21) Appl. No.: 12/745,679

(22) PCT Filed: Dec. 19, 2008
(86) PCT No.: PCT/EP08/10892

§ 371 Date: Jun. 1, 2010

Related U.S. Application Data
(60) Provisional application No. 61/016,891, filed on Dec. 27, 2007.

(51) Int. Cl. A61K 31/138; A61P 9/00

(52) U.S. Cl. 514/652

(57) ABSTRACT

The present invention relates to a method of reversing the electrophysiological cardiac remodeling of animals with heart disease. More specifically, the method includes administering to an animal in need thereof a β-adrenoceptor blocker.

### FIELD OF THE INVENTION

The present invention relates to a method of reversing the electrophysiological cardiac remodeling of animals with heart disease with the use of β-adrenoceptor blockers.

### BACKGROUND OF THE INVENTION

β-adrenoceptor blockers are known to exert positive effect on the cardiovascular system mainly through the blockade of cardioselective β1-receptors. A number of different β-adrenoceptor blockers, such as propranolol, atenolol, metoprolol, carvedilol, and bisoprolol, are approved for treatment of human cardiovascular disease. Due to their negative inotrope and chronotrop effects β-blockers directly improve the hemodynamic economics of the heart's work load. The β-blockers are used in humans for treatment of stable chronic heart failure with limited systolic function, tachyarrhythmia, hyperkinetic heart syndrome, as well as for treatment of hypertension, coronary artery disease (CAD) and prophylaxis of heart attack.

In the dog, Chronic Valvular Heart Disease (CVHD), also known as mitral regurgitation (MR), is the most common cardiovascular disease, accounting for approximately 75% of all cases of cardiovascular disease in dogs. The disease is highly correlated to age, and typically occurs in smaller breeds such as Cavalier King Charles Spaniels, Poodles, Chihuahuas, Fox Terriers, and Dachshounds. The pathogenesis of this cardiovascular disease may be seen to include three major phases. In the first phase there is injury to the heart, but in many cases it is unrecognized and asymptomatic. In the second phase, there is compensation of the progressed initial injury to ensure cardiac output by activation of the sympathetic nervous system (increase of heart rate=positive chronotropy, conduction rate=positive dromotropy and increased contractility=positive inotropy), and the renin-angiotensin-aldosterone system (RAAS) as well as by elaboration of a variety of cytokines. This phase is usually characterized by signs of heart disease, such as cardiomegaly or heart murmur, and is diagnostically evident, by echocardiography or thoratic radiographs, but is clinically asymptomatic. In the third phase, there is an onset of heart failure. In this phase there is inadequate cardiac output due to failure of the chronic compensation mechanisms (increased sympathetic activation), characterized by clinical symptoms like exercise intolerance, cough and dyspnea due to pulmonary edema or effusion subsequent to pulmonary congestion.

Currently there are clinical studies with angiotensin-converting enzyme (ACE) inhibitors and calcium sensitizers for phase one and phase two, however, these drugs do not show signs of reversing the electrophysiological cardiac remodeling of animals with heart disease. It is also believed that a treatment for phase one could consist of a repair of the initial injury or underlying molecular mechanisms, i.e. reverse or slow down cardiac remodeling, however such repair is currently unknown. The typical treatment for phase three, symptomatic heart failure, consists of diuretic therapy, to resolve, for example, pulmonary edema, and a reduction of afterload (increase of cardiac output) by an ACE inhibitor (peripheral vasodilation). Digitalis glycosides, such as digoxin, are given in cases of atrial fibrillation or if a positive inotropy is needed. β-blockers have also been used to treat dogs in heart failure. These treatment regimes, with diuretics and ACE inhibitors, have been known to cause several problems for the dogs. First, it is difficult to define the exact dose of diuretic required for each dog. Once defined the dose is often close to a dose that might result in electrolyte disturbance, dehydration, and development of pre-renal azotemia. The combined use of ACE inhibitors and diuretics compromises one of the kidneys' normal compensatory mechanisms (vasoconstriction of the efferent arteriole) and can lead to elevation of BUN and creatinine if an excessive diuretic does is initiated. Although β-blockers provided some benefits, such as up regulation of previously down regulated beta-receptors and improved cardiac performance, the benefits are not seen for several months. Finally, even with these treatments, the average survival of dogs after the onset of heart failure, phase three, is comparatively short.

As such, there is a need for a method of treating dogs in phase two such that phase three, the onset of heart failure, is delayed or prevented. In particular, there is a need for a method of reversing the electrophysiological cardiac remodeling of dogs with heart disease.

### DETAILED DESCRIPTION OF THE PREFERRED EMBODIMENTS

Advantageously, the present invention provides a method of reversing the electrophysiological cardiac remodeling of dogs with heart disease.

I. Electrophysiological Cardiac Remodeling

Chronic Valvular Heart Disease (CVHD) is caused by a progressive myxomatous degeneration of the atrioventricular (AV) valves. As described above, the cardiovascular disease may be seen to include three major phases. In the first phase there is an initial injury to the AV valves, but it is typically unrecognized and asymptomatic. In phase two, the compensatory mechanisms, the sympathetic nervous system (SNS), of the body are initially supportive; but long-term activation of the SNS exerts deleterious effects that ultimately damage the heart and lead to heart failure. The SNS tries to compensate for the injury by increasing the heart rate, conduction rate, and contractility, and the RAAS as well as by elaboration of a variety of cytokines. Norepinephrine (NE) is the primary signaling molecule of cardiac adrenergic activity at this stage and is a powerful mediator of cardiotoxicity (pathologic myocardial damage), cardiac hypertrophy, and a strong activator of apoptosis. An increased sympathetic drive is also responsible for eccentric hypertrophy of cardiac areas, leading to left ventricular hypertrophy and chamber dilation, increased cardiac mass, fiber slippage, loss of interstitial collagen and changes in the electrophysiology in dogs with heart disease. All these adaptive processes, which are from the physiological perspective pathological and are characterized through an altered action of the heart, in particular by an altered shape of the curve and duration of the action potentials and changes in potassium currents across cell membranes of the myocardium, are termed electrophysiological cardiac remodeling as used herein.

Typically once the heart has been remodeled this is the final common pathway to heart failure, phase three, whether initiated by pressure or volume overload. Left ventricular dysfunction, enlargement of atria and ventricles, increase in cardiac mass, contractile dysfunction and collagen loss were observed in experimentally induced MR in dogs, and finally resulted in symptomatic heart failure and death.

II. β-adrenoceptor Blocker

The method of reversing the electrophysiological cardiac remodeling of animals with heart disease includes administering to an animal, in need thereof, an effective amount of a β-adrenoceptor blocker, a pharmaceutically acceptable derivate or salt thereof, or mixtures thereof.

The term “β-adrenoceptor blocker” or “β-blocker” as used herein refers to beta-adreno receptor blockers (“beta blockers”), which competitively and reversably bind to β-adrenergic receptors. When bound to the β-adrenergic receptors, the β-blockers prevent the adrenergic stimulation through endogenous catecholamines (epinephrine (adrenaline) and norepinephrine (noradrenaline)) in particular.

The β-blockers are negative inotrops (reduce myocardial contractility), negative chronotrops (reduce heart rate), negative dromotrops (reduce atrial-ventricular conduction rate), and positive lusitrops (support relaxation of the myocard). By this action β-blockers suspend the circulus virtuosus derived from constantly elevated deleterious endogenous catecholamine levels, which mediate a constant “fight or flight” response.

Suitable β-adrenoceptor blockers include propanolol, metoprolol, atenolol, bisoprolol, pindolol, alprenolol, carvedilol, acebutolol, betaxolol, esmolol, nebivolol, CGP 20712, SR 59230A, CGP-12177, ICI 118551, pharmaceutically acceptable salts, derivates, metabolites, pro-drugs, and combinations thereof. In one embodiment, the β-blocker may be bisoprolol, a pharmaceutically acceptable salt, derivate, metabolite, pro-drug, or combinations thereof. In another embodiment, the β-blocker may be bisoprolol fumarate. Bisoprolol fumarate corresponds to the formula (I):

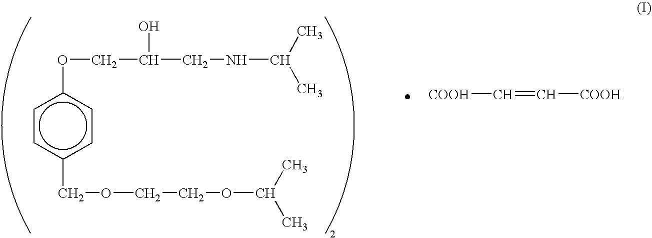

Bisoprolol fumarate may be purchased commercially from Merck KgA, Darmstadt, Germany (EMD Pharmaceuticals in the US) or made in accordance with methods generally known in the art.

The β-blocker may be administered by itself or it may also be administered as part of a formulation. The formulation may be a solid, gas, or liquid formulation. In one embodiment, the formulation is a liquid formulation. In another embodiment, the liquid formulation may include from about 0.001% to about 1% by weight β-blockers, from about 40% to about 80% by weight of a solvent, such as water, and from about 1% to about 70% by weight of a thickener, such as glycerine or hydroxypropyl methylcellulose. The formulation may also include other ingredients such as preservatives, solvents, and flavorings, among others. In another embodiment, the formulation may be, for example, as detailed in PCT Publication WO 2007/124869, which is hereby incorporated by reference in its entirety. In yet another embodiment, the formulation may include from about 0.01 to about 0.5% by weight bisoprolol fumarate.

The β-blockers of the present invention are administered in an effective amount to reverse the electrophysiological cardiac remodeling of dogs with heart disease. In one embodiment, the β-blockers are administered once a day. In another embodiment, the β-blockers are administered multiple times a day. In yet another embodiment, the β-blockers are administered at a dose of from about 0.001 mg/kg to about 100 mg/kg. In a further embodiment, the β-blockers are administered at a dose of from about 0.001 mg/kg to about 10 mg/kg. In another embodiment, the β-blockers are administered at a dose of from about 0.001 mg/kg to about 1 mg/kg.

The β-blockers may be administered in the form of, for example, tablets, capsules, solutions, gel capsules, pastes. In one embodiment, the β-blockers may be administered in the form of an oral solution. Alternatively, the β-blockers may be administered by parenteral administration, such as, for example, by injection (intramuscular, subcutaneous, intravenous, intraperitoneal and the like), implants, or by nasal administration.

The β-blockers may be administered once or in multiple doses. Alternatively, the β-blockers may be administered continuously as necessary throughout the day.

Animals having heart disease whose electrophysiological cardiac remodeling may be reversed include farm animals, such as cattle, horses, sheep, pigs, goats, camels, water buffalo, donkeys, rabbits, fallow deer, reindeer, furbearing animals such as mink, chinchilla, raccoons, birds, such as chickens, geese, turkeys, ducks, pigeons, species of birds intended to be kept in the home and in zoos, and also fish. Other animals include laboratory and experimental animals, such as mice, rats, guinea pigs, hamsters, dogs, cats, and MUMS (minor use and minor species). Yet other animals include pets and hobby animals, such as rabbits, hamsters, guinea pigs, mice, horses, reptiles, corresponding species of birds, dogs, and cats. In one embodiment, the animal is a dog.

II. Reversal of Electrophysiological Cardiac Remodeling

There are several ways to measure the electrophysiological cardiac remodeling of the heart including the action potential of the myocytes of the heart and the potassium current, among others. The action potential duration may be measured at 50% repolarization and at 90% repolarization. There are two potassium currents that modulate the resting membrane potential and the action potential duration, the inward rectifier potassium current, and the transient outward potassium current. The inward rectifier potassium current (IK1) is the primary determinant of the resting membrane potential (inward current) and modulates the final phase of repolarization (outward current). Reduction in inward current result in depolarization of the resting potential, while reductions in the outward current may contribute to action potential duration prolongation.

An animal without heart disease will have an action potential duration (ADP) of about 300-400 ms and about 400-500 ms, respectively (ADP 50% and ADP 90% respectively, measured at 0.5-1 Hz). An animal with heart disease/heart failure, that has undergone electrophysiological cardiac remodeling, shows an action potential duration of about 400-500 ms and about 500-700 ms, respectively (ADP 50% and ADP 90%, respectively measured at 0.5-1 Hz). Under administration of an effective dose of a β-blocker, the action potential duration will be reversed back to a length of a non-injured heart of about 300-400 ms and about 400-500 ms, respectively (ADP 50% and ADP 90% respectively, measured at 0.5-1 Hz).

Once an animal that has heart disease/heart failure is administered an effective dose of a β-blocker, the peak outward current increases from about 1.25 to about 2.0 (Iₖ₁ (pKa/pF). This leads to the normalization of the current conductance of the dog's heart myocytes.

### DEFINITIONS

To facilitate understanding of the invention, a number of terms and abbreviations as used herein are defined below:

The term “CVHD” refers to chronic valvular heart disease.

The term “DCM” refers to dilated cardiomyopathy.

The term “MR” refers to mitral regurgitation.

The term “CAD” refers to coronary artery disease.

The term “heart disease” as used herein refers to a heart condition prior to the onset of cardiac insufficiency or heart failure.

The term “β-adrenoceptor blocker” or “β-blocker” as used herein refers to beta-adreno receptor blockers (“beta blockers”), which competitively and reversably bind to β-adrenergic receptors. When bound to the β-adrenergic receptors, the β-blockers prevent the adrenergic stimulation through endogenous catecholamines (epinephrine (adrenaline) and norepinephrine (noradrenaline)) in particular.

### EXAMPLES

The following examples illustrate various embodiments of the invention.

### Example 1

A study was conducted with two groups of conscious dogs with pacing-induced heart failure to determine the tolerance and potential effects of different doses of bisoprolol fumarate. This data was compared to historical data from untreated normal dogs without induced heart failure. ECG (PQ, QRS, RR, QT, QTcF, and QTcV intervals), echocardiography (left ventricular shortening fraction (LVSF) and systemic arterial blood pressure (SBP, DBP, MAP and pulse pressure) were monitored in the two groups. Heart failure was produced by rapid ventricular pacing to reduce left ventricular shortening fraction (LVSF) greater than 15% from baseline.

In the first group, the conservative up-titration study, the dogs were treated with weekly increasing oral doses of 0.005, 0.01, 0.03, 0.05 and 0.1 mg/kg bisoprolol fumarate. In the second group, the aggressive up-titration study, the dogs were treated with weekly increasing doses of 0.01, 0.05, 0.1, 0.5 and 1 mg/kg bisoprolol fumarate on top of a dose of 0.5 mg/kg of enalapril, 4 mg/kg of furosemide, and 0.003 mg/kg of digoxin. These two groups were compared to a placebo group that was treated with the same doses of the standard heart failure therapy (enalapril, furosemide and digoxin) alone.

Results of this study indicate that the oral solution of bisoprolol fumarate was well tolerated in dogs with pacing-induced heart failure, even at doses that exceed anticipated target treatment doses.

The doses used in both groups provided both the possibility to safely initiate β-blocker therapy with bisoprolol at a low dose that is increased slowly, as well as a dose with a near to maximum cardioselective β-blockade effect (prolongation of PQ interval and reduction of heart rate) in dogs with heart failure.

After altogether 5 weeks of treatment the dogs were anaesthetized according to standard veterinary procedures and ex vivo ventricular myocytes were directly isolated from the mid-lateral left ventricular free wall using an isolation procedure described by Kubalova et al., which results in isolation of myocytes from the midmyocardial region. Afterwards the animals were humanely euthanized. Recordings of single cell action potentials and K+ currents were made. See Kubalova et al., Abnormal intrastore calcium signaling in chronic heart failure, Proc Nat Acad Sci 2005; 102: 14104-14109.

For measurement of the action potentials myocytes were placed in a laminin coated cell chamber and superfused with a bath solution. Only quiescent myocytes with sharp margins and clear striations were used for the electrophysiological study. Borosilicate glass micropipettes were filled with a pipette solution that was pH adjusted to 7.2. Perforated whole cell patch clamp was used to minimize alterations in the intracellular milieu. Action Potentials (APs) were recorded with the perforated whole cell patch techniques. Action potentials were recorded in isolated ventricular myocytes, which were characterized in the standard manner as the durations to 50% and 90% of repolarization. APs were measured as the average of the last 10 (steady-state) APs, obtained during a train of twenty five APs at each stimulation rate. An average of 2-3 myocytes was measured from each heart failure dog.

Action Potentials were recorded in four groups. The following numbers of recordings were obtained and used in the analyzed data (number (n) indicates the number of myocytes):

Control (CTRL, untreated, healthy dogs) (n=10) HF-placebo (placebo-treated dogs in heart failure (HF-PL)) (n=17)

HF-C-Up bisoprolol, according to conservative up-titration bisoprolol treated dogs in heart failure (n=13)

HF-A-Up bisoprolol, according to aggressive up-titration bisoprolol treated dogs in heart failure (n=15)

Resting membrane potential was measured at 0.5 Hz and 1 Hz to bracket the physiologic range of resting heart rates (FIG. 1).

Resting membrane potentials (FIG. 1) do not differ between groups, there were no significant differences in resting membrane potentials at 0.5 and 1 Hz. All groups had an average resting potential of at least −75 mV, which is consistent with normal values in isolated myocytes. See Szentadrassy et al., Apico-basal inhomogeneity in distribution of ion channels in canine and human ventricular myocardium, Cardiovasc Res 2005; 65: 851-860.

The action potential duration (APD) at 50% repolarization (APD50, FIG. 2) was significantly prolonged in the heart failure-placebo treated group at 0.5 Hz and 1 Hz compared to normal control values.

At 0.5 and 1 Hz a statistically significant reduction in APD50 was seen with doses of bisoprolol used in both the conservative (HF-C-Up) and aggressive (HF-A-Up) up titration protocol groups compared to the placebo-treated heart failure group. Values in the bisoprolol treated groups did not differ from APD50 measured in normal control myocytes.

The action potential duration at 90% repolarization (APD90, FIG. 3) was significantly prolonged in the heart failure-placebo treated groups at 0.5 and 1 Hz compared to normal control values.

At 0.5 and 1 Hz, the conservatively and aggressively up-titrated bisoprolol treatment groups (HF-C-Up and HF-A-Up) significantly attenuated the heart failure induced prolongation of the APD90, to values that did not differ from normal controls.

### Summary of HF-Induced Changes in Action Potentials

The HF-induced changes in the action potential durations (particularly APD90 prolongation which is known to correspond to increased arrhythmia risk—specifically drug-induced Torsades de Pointes) at physiologically relevant heart rates during β-adrenergic blockade (in humans the target heart rate is often around 60 BPM or 1 Hz) are significantly attenuated and even reversed to the physiological normal by doses of bisoprolol used with both, the conservative and aggressive up-titration dosing regimens.

There are two K+ currents which are expected to modulate the resting membrane potential and the action potential duration, and are known to be altered during heart failure, the inward and the outward K+ currents.

The inward rectifier K+ current (I_{K1}) is the primary determinant of the resting membrane potential (inward current) and modulates the final phase of repolarization (outward current). Reductions in inward current result in depolarization of the resting potential, while reductions in outward current can contribute to action potential duration prolongation.

I_{K1} was recorded in each of the four groups, data was recorded and analyzed. Average current density-voltage relationships are shown in FIG. 4.

No statistical difference was found for the inward I_{K1} current conductance between the groups (FIG. 5 top). However, with the doses used in the aggressive up-titration protocol a trend to a lower slope conductancy can be observed, which if of sufficient magnitude could contribute to an undesirable destabilization of the resting membrane potential.

The peak outward K+ current was recorded in each of the four groups (FIG. 5 bottom), data was recorded and analyzed.

The peak outward I_{K1} current (FIG. 5 bottom) was increased in the HF-C-Up group relative to both, the placebo (HF PL) and aggressive up-titration protocol (HF-A-Up) bisoprolol group.

The transient outward K+ current, (Iₜₒ) was recorded in all four groups and data was recorded and analyzed. Average current density-voltage relationships are shown in FIG. 6.

Heart failure reduced Iₜₒ at all test voltages compared to control (p<0.05). Bisoprolol doses as used with the aggressive up-titration protocol (HF-A-Up Bis) did not alter the effects of heart failure on Ito, whereas at the two highest test potentials (+40 and +50 mV), bisoprolol doses used with the conservative up-titration protocol (HF-C-Up Bis) significantly attenuated heart failure induced reductions in Iₜₒ.

### Summary on HF-Induced Changes in K+ Currents

In summary it can be stated that no difference was found in the inward I_{K1}, current conductance between the groups (FIG. 5 top), which is an indicator for a stable resting membrane potential under treatment with bisoprolol.

The peak outward current (FIG. 5, bottom) was increased at doses used with the conservative up-titration protocol group relative to the placebo group with dogs in heart failure. This would suggest a potentially beneficial effect of bisoprolol fumarate on terminal repolarization to normalize repolarization in dogs with heart failure.

Heart failure induced reductions in the transient outward K+ current Iₜₒ were significantly attenuated in the conservative up-titration protocol bisoprolol-treated group.

The model used for this examination is an acute model with a rapid onset of heart disease. Under normal conditions, within the patient, this pathological process generally has a much more prolonged time of onset.

Electrophysiology and the electromechanical linkage of electrophysiology/membrane potentials and cardiac contraction are the central physiological aspect of hemodynamics and heart function. This makes it most likely that the observed properties of bisoprolol are highly beneficial in case of prevention and/or therapy of heart disease and heart failure in dogs.

What is claimed is:

1. A method of reversing the electrophysiological cardiac remodeling of an animal with heart disease, the method comprising administering to the animal in need thereof an effective amount of a β-adrenoceptor blocker.

2. The method of claim 1, wherein the β-adrenoceptor blocker is selected from the group consisting of propanolol, metoprolol, atenolol, bisoprolol, pindolol, alprenolol, carvedilol, acebutolol, betaxolol, esmolol, nebivolol, CGP 20712, SR 59230A, CGP-12177, ICI 118551, pharmaceutically acceptable salts, derivates, metabolites, pro-drugs, and combinations thereof.

3. The method of claim 2, wherein the β-adrenoceptor blocker is bisoprolol.

4. The method of claim 1, wherein the β-adrenoceptor blocker is bisoprolol fumarate.

5. The method of claim 1, wherein the animal is a dog.

6. The method of claim 1, wherein the effective amount of β-adrenoceptor blocker is from about 0.001 mg/kg to about 1 mg/kg.

7. A method of reversing the electrophysiological cardiac remodeling of animals with heart disease, the method comprising administering to an animal in need thereof an effective amount of a β-adrenoceptor blocker formulation.

8. The method of claim 7, wherein the formulation is an oral formulation.

9. The method of claim 8, wherein the formulation comprises:
a. From about 0.001% to 1% by weight of a β-blocker;
b. At least about 40% by weight of a solvent; and,
c. From about 1 to about 70% by weight of a thickner.

10. The method of claim 9, wherein the β-blocker is bisoprolol fumarate, wherein the solvent is water, and wherein the thickener is hydroxypropyl methylcellulose.

11. The method of claim 7, wherein the animal is a dog.
